(19) United States
(12) Patent Application Publication
(10) Pub. No.: US 2009/0098179 A1
(43) Pub. Date: Apr. 16, 2009

(54) FLUID EQUILBRATED ABSORBENT POLYMERIC MATERIALS, DEVICES INCLUDING SAME AND PACKAGING FOR SAME

(76) Inventors: Matthew R. Gevaert, Central, SC (US); Martine LaBerge, Seneca, SC (US); Jianrong Feng, Burlington (CA)

Correspondence Address: DORITY & MANNING, P.A., POST OFFICE BOX 1449, GREENVILLE, SC, 29602-1449, US

(21) Appl. No.: 12/065,783

(22) PCT Filed: Sep. 27, 2006
(86) PCT No.: PCT/US06/37900

§ 371 Date: Oct. 2, 2008

Related U.S. Application Data
(60) Provisional application No. 60/720,839, filed on Sep. 27, 2005.

(51) Int. Cl. A61F 2/00

(52) U.S. Cl. 424/423; 523/113

(57) ABSTRACT

Disclosed are methods and devices for the engineering, formation, and post-formation handling of absorbent polymeric materials. Devices intended for use in a fluid environment that include an absorbent polymeric material can be formed and processed to ensure that upon installation of the device, the polymeric material can not only have the desired physical characteristics for the intended use, but can also be pre-equilibrated for the environment in which the device will be utilized. In particular, the polymeric materials can be formed with particular characteristics such that, upon absorption of a fluid, the characteristics will alter in a predetermined way to provide the materials within target specifications during use. In addition, the materials can be stored in a fluid so as to exhibit the desired operating characteristics immediately upon installation. In one particular embodiment, the invention is directed to biocompatible, implantable devices designed for use in vivo.

### CROSS REFERENCE TO RELATED APPLICATIONS

This application claims benefit to U.S. Provisional Application Ser. No. 60/720,839 filed Sep. 27, 2005.

### BACKGROUND OF THE INVENTION

Polymeric materials offer many advantages for use in a wide variety of applications due to their unique characteristics. For example, polymeric materials can be formed with excellent hardness and strength characteristics while maintaining deformability and resistance to degradation and thus can be beneficially utilized in physically demanding, e.g., load bearing, applications. Many polymers are also at least somewhat absorbent with the physical and mechanical characteristics of the materials often changing upon the fluid absorption. For example, mechanical characteristics such as hardness and tensile strength can be affected upon fluid absorption by polymeric materials. In addition, the physical dimensions of a polymeric piece can change upon fluid absorption, changing the tolerance values and/or contact characteristics between that part and another, for instance in a joint or bearing application. As such, many polymers have not reached their greatest potential for use in fluid environments, due at least in part to the absorbent characteristics of the materials.

What is needed in the art are methods and devices for better preparing polymeric materials for use in fluid environments, not only preventing “break-in” periods for materials following installation or implantation, but also paving the way for novel applications for certain polymeric materials not before considered suitable for use in fluid environments.

### SUMMARY OF THE INVENTION

In one embodiment, the present invention is directed to a method for processing a device prior to use in a fluid environment. The method can include storing a device in a sealed container. The device can include an absorptive polymeric material that includes a synthetic polymer such as, for example, an engineering polymer or a step-growth addition polymer. In conjunction with the device, the container holds a fluid. The polymeric material is capable of absorbing an amount of this fluid. For instance, the polymeric material can absorb an amount of this fluid that is less than about 100% of the weight of the polymeric material.

During storage in the container, the polymeric material can absorb at least a portion of the fluid. This absorption can lead to characteristic change in the polymeric material. For instance, a dimensional characteristic, one or more mechanical characteristics, or one or more electrical characteristics of the polymeric material can change upon absorption of the fluid. Optionally, the fluid held in the container can also include an additive. For instance, the fluid can be a fluid mixture including more than one type of fluid, and the polymeric material can absorb components of the fluid mixture during storage.

In one particular embodiment, the device can be an implantable device. Accordingly, the fluid in which the device can be stored can be an appropriate aqueous fluid. e.g., a simulated body fluid. Optionally, the fluid can include a biologically active additive, such as a drug, a nutrient, an antibacterial agent, or the like.

The present invention is also directed to methods for forming devices. For instance, during formation of a device, the polymeric material can be formed such that a characteristic of the material falls outside of the specifications required during use in the intended fluid environment. During the storage process, the absorption of the storage fluid by the polymeric material can cause the characteristic to change in a predetermined fashion, such that the fluid-equilibrated polymeric material can alter to exhibit the characteristic within the desired specification range.

In another embodiment, the invention is directed to a packaging system for a device such as described herein. The system can include a sealable container, a fluid held within the container, and the device.

Fluids of the packaging system can be any suitable fluid for pre-soaking the device and bringing about the desired fluid equilibration. For instance, the fluid can be aqueous or organic. Similarly, the fluid can be a liquid, a gas, or a vapor.

### BRIEF DESCRIPTION OF THE FIGURES

A full and enabling disclosure of the present invention, including the best mode thereof to one of ordinary skill in the art, is set forth more particularly in the remainder of the specification, including reference to the accompanying figures in which:

FIG. 1 illustrates the changes in mechanical properties of a polyurethane over time following immersion and equilibration in water; and

FIG. 2 illustrates the dimensional changes of polyurethane bearing tubes following immersion and equilibration in water.

### DEFINITIONS

According to the present disclosure, the term ‘fluid environment’ is herein defined to refer to an environment that is primarily comprised of a fluid other than air. For example, while the term encompasses an environment in which a device is completely and continually immersed in or surrounded by a particular fluid, the term also encompasses an environment in which a device can be maintained in an essentially saturated or fluid equilibrated state with a fluid that can be the primary constituent of the environment. In addition, a fluid environment can include any fluid including liquid as well as vaporous and/or gaseous fluids including steam, carbon dioxide, nitrogen, and the like, as well as mixtures of such.

For purposes of this disclosure, the term “absorbent polymeric materials” is herein defined to refer to polymeric materials that can absorb an amount of a fluid. For instance, an absorbent polymeric material of the invention can absorb less than about 100% of the weight of the polymeric material.

### DETAILED DESCRIPTION OF THE INVENTION

Reference will now be made in detail to various embodiments of the invention. Each example is provided by way of explanation of the invention, not limitation of the invention. In fact, it will be apparent to those skilled in the art that various modifications and variations can be made in the present invention without departing from the scope or spirit of the invention. For instance, features illustrated or described as part of one embodiment, can be used on another embodiment to yield a still further embodiment.

In general, the present invention is directed to the recognition and optimization of changes in characteristics of polymeric materials upon fluid absorption. More particularly, the present invention is directed to methods and devices that can accommodate for those expected changes during formation and/or processing of the materials so as to better utilize the materials in fluid environments. For instance, the methods and devices of the present invention can be utilized during the engineering, formation, storage, shipping, and/or handling of devices comprising absorbent polymeric materials so as to provide devices that can be beneficially utilized in a fluid environment.

More specifically, the invention is directed to the engineering, formation and/or storage of absorbent polymeric materials that exhibit a change in one or more characteristic of the material upon fluid absorption. For instance, the polymeric material can exhibit change in electrical, physical and/or mechanical characteristics upon fluid absorption.

Devices suitable for treatment and/or handling according to the present invention include devices that can be utilized in a fluid environment and include as at least a portion of the device an absorbent polymeric material as herein defined. In one particular embodiment, the devices can be biomedical and/or biocompatible devices. In other embodiments, however, the devices can be intended for other fluid environments including either aqueous or organic fluids in industrial applications. For example, the devices can be intended for use in industrial applications packed in oil or grease. Devices that can be processed as herein described can also be those intended for use in physically demanding, e.g., load bearing applications.

Devices of the invention can be formed exclusively of one or more absorbent polymeric materials as herein described or optionally can be composite devices that include absorbent polymeric materials as well as other materials. For example, the devices can include absorbent polymeric materials in conjunction with one or more materials that do not absorb the fluid of the fluid environment, for example non-absorbent materials such as metals or non-absorbent polymeric materials.

In one embodiment, the devices can include an absorbent polymeric material in conjunction with one or more naturally occurring biocompatible materials. For example, a portion of the device can include naturally occurring materials that may or may not be absorbent. In one particular embodiment, a device can include one or more biological tissues. One example of such a device is an implantable device such as an artificial heart valve including valve leaflets formed of natural tissues, for example allograft or xenograft tissue. The device can include an absorbent polymeric material as herein described in combination with the natural tissue, for example as a polymeric stent surrounding the valve leaflets. In another embodiment, the device can include one or more absorbent polymeric materials in conjunction with natural bone mineral, for instance for use in an in vivo implantation of a bone graft or scaffolding. The devices can optionally include synthetic biocompatible materials such as artificial tissue material or synthetic bone mineral.

Other exemplary materials that can be combined with the absorbent polymeric materials to form devices as herein described can include materials that are highly absorbent. For example, a device can be intended for use in an aqueous environment and can include an absorbent polymeric material in conjunction with one or more phases of a highly absorbent colloid such as a hydrogel. Highly absorbent colloids are multi-phase systems including a dispersed phase that can be distributed in a continuous phase. While one or more of the phases of a highly absorbent colloid can be three-dimensional or polymeric in nature, these materials differ from the absorbent polymers described herein at least in the large absorbency capability of the highly absorbent colloid systems. For instance, when considering a hydrogel in fluid equilibrium in water, the amount of water absorbed by and dispersed within the continuous phase of the colloid can be many times the weight of the continuous phase. Other exemplary highly absorbent materials that can be combined with the absorbent polymeric materials can include materials having extremely high surface areas such as foams, activated carbons, nanostructures or composites thereof, and the like.

The polymeric materials of the devices can include other additives as are generally known in the art. For example, the polymeric materials can include additives as are generally known in the art such as coloring agents, anti-static agents, conductive agents, antioxidant agents, antimicrobial agents, adhesion agents, stabilizers, plasticizers, brightening compounds, clarifying agents, ultraviolet light stabilizing agents, surface active agents, odor enhancing or preventative agents, light scattering agents, halogen scavengers, and the like. The polymeric materials may contain any additives or fibers, for instance, in one embodiment the polymeric materials can be composite materials including a polymer matrix and one or more additives such as reinforcement fibers or structures encapsulated or otherwise contained in the matrix.

The absorbent polymeric materials of the invention can generally include at least one synthetic polymer. The polymeric materials can include other components as well, however, in addition to a synthetic polymer. For example, the polymeric material can include a synthetic polymer combined with polymers that can be either natural or synthetic. Polymers of the polymeric materials can include any polymer with fluid absorbing characteristics to be used in fluid environment while retaining the solid state.

The synthetic polymers of the invention can be homopolymers, random copolymers, block copolymers, functionalized polymers, cross-linked polymers, blends, thermosets, thermoplastics, combinations thereof, or any other synthetic polymer as described herein that can be utilized to form a polymeric material that is at least somewhat absorbent. For example, the synthetic polymer can be a polymer or copolymer including a polyester, a polyether, a polycarbonate, a polyamine, a polyurea, a polyurethane, a polyvinyl, and the like.

In one embodiment, the polymeric materials can include one or more engineering polymers. A non-limiting exemplary list of engineering polymers suitable for the disclosed polymeric materials can include polyolefins, polyurethanes, silicones, fluoropolymers, polyesters, polyoxymethylene, polyamides, polyimides, polyamide-imides, polycarbonates, polysulfones, polyphenylene sulfides, polyketones, polybenzimidazole, epoxies, phenolics, bismaleimide, and so on.

In one embodiment, the polymeric materials can include at least one polymer formed according to a step-growth polymerization process. For example, the polymeric material can include at least one step-growth polymerization polymer such as, but not limited to, polyurethanes, polyesters, polyoxymethylene, polyamides, polyimides, polyamide-imides, polycarbonates, polysulfones, polyphenylene sulfides, polyketones, polybenzimidazole, epoxies, phenolics, bismaleimide, and so on.

The absorptive polymeric materials of the present invention include those capable of absorbing an amount of a fluid less than about 100% of the weight of the polymer. For example, in one embodiment, the absorbent polymeric materials can be capable of absorbing an amount of a fluid that is less than about 75% of the weight of the polymeric material. In other embodiments, the maximum fluid absorption capability of the polymeric materials can be less, for instance, less than about 20% of the weight of the polymeric material, less than about 10%, less than about 5%, or less than about 1% of the weight of the polymeric material, in certain embodiments.

The polymeric materials of the devices can generally exhibit variation in one or more material characteristics upon fluid absorption. For example, the polymeric material can exhibit a change in electrical, mechanical, and/or physical characteristics. The devices including the polymeric materials can be intended for use in a fluid environment, and the prior fluid-equilibration processing of the devices can accommodate for expected and characteristic changes that occur to the polymeric portion of the device upon fluid absorption. In this way, the device can be fluid equilibrized prior to implantation or installation.

For example, a device of the invention can be intended for use in an application requiring a very exacting tolerance junction between the absorbent polymeric material and an adjacent material, for instance as a bearing. Moreover, the polymeric material can be one that can exhibit a dimensional change upon fluid absorption. According to one such embodiment, the polymeric material of the device can be engineered and formed to a predetermined dry size and then held prior installation in a fluid environment such that the polymeric material can become fluid equilibrated and stabilized at the new size and/or shape that is different from the originally formed dry shape and/or size. Accordingly, upon installation, the polymeric material can already be stabilized with the desired dimensions and the bearing junctions can exhibit the desired tolerance.

A device including an absorbent polymeric material as herein described can be intended for use in a fluid environment in a load bearing application. Accordingly, the device specification can require certain wear and strength characteristics. In addition, the polymeric material of the device can exhibit change in mechanical characteristics upon absorption of a fluid. For example, upon absorption of a fluid, the polymeric material may become appreciably softer. According to such an embodiment, the process of engineering and forming the device can include designing the polymeric material in the dry state with mechanical characteristics greater than those that will be required when the piece will be used in the fluid environment, i.e., engineered and designed outside of the specification range. Upon storing the device in a fluid and absorption of an amount of the fluid, the polymeric material can alter as expected (e.g., soften) and can exhibit mechanical characteristics in the specified range following that characteristic alteration. In other words, the process of forming the device can include “over-engineering” the material, i.e., designing and forming the material with characteristics outside of those specified for the intended application, so as to accommodate the expected changes to the material upon location in a fluid environment.

In one particular embodiment, the device can include at least a portion formed of a polyurethane. Polyurethanes can be formed to exhibit many desirable characteristics, depending upon the desired application. For example, polyurethanes can be formed to be quite hard and suitable for use in load bearing applications. Alternatively, polyurethanes can be formed to be soft and pliable and as such are often used to form devices such as tubing. In addition, polyurethanes can be biocompatible, and thus suitable for use in many biologically based applications, such as catheters, pace maker leads, artificial hearts, and the like. Harder polyurethanes have also been considered for use in biological applications. For example, co-owned U.S. Patent Application Publication No. 2006/0178497 to Gevaert, et al., which is incorporated herein in its entirety by reference, discloses implantable devices including hard biocompatible polyurethanes.

Changes of mechanical properties due to fluid absorption, and in particular due to liquid absorption, can be quite pronounced in polyurethanes. As such, material design concepts based upon properties of polyurethanes in the dry state and at similar levels as less absorptive or nonabsorptive polymers such as ultra-high molecular weight polyethylene may lead to poor performance or failure in a fluid state. In addition, examination of material performances under testing conditions closer to those expected in the fluid environment of use has not been a wide area of work. Such examination could prove beneficial, however, as it could indicate problems with design concepts for absorptive materials for targeted applications in a fluid environment, particularly when the materials have been engineered and designed according to performance characteristics in the dry state. The presently disclosed methods and devices address these and other problems when considering absorptive materials for applications in fluid environments.

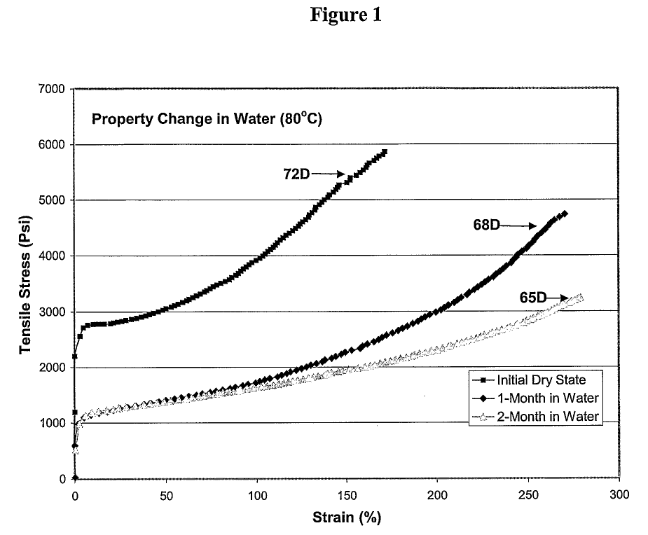

For example, and with reference to FIG. 1, the tensile and hardness properties of an exemplary absorptive polyurethane are shown. As can be seen, in the dry state the polyurethane material had an initial hardness of 72D with tensile strength and elongation characteristics as shown in FIG. 1. Following immersion in water at 80° C. for one month, however, the hardness of the material dropped to 68D, with a drop to a hardness of 65D after another month in the 80° C. water. The tensile strength and elongation characteristics of the material also exhibited significant changes upon the water immersion, as can be seen. While the effects of the water immersion in this case may be exaggerated by the relatively high soaking temperature, for instance as compared to biological temperatures, these conditions are useful to show behavioral trends for lower temperatures in shortened time periods, and the results clearly illustrate that the effects of water absorption on polyurethanes can be beneficially considered in order to better design and fabricate such materials for use in fluid environments.

When preparing absorptive polymeric materials for use in fluid environments, and in particular in fluid environment applications in which particular characteristics of the materials can be of paramount importance, such as load-bearing applications, the materials can be engineered and formed so as to exhibit the property characteristics, e.g., modulus, tensile strength, elasticity, joint tolerance, etc., that will be required following equilibration in the fluid environment as opposed to in the dry state. Accordingly, in the present invention, the absorbent polymeric materials can be formed so as to meet the property specifications and requirements following fluid equilibration and thus at the expected conditions during use.

In addition to engineering and forming absorbent polymeric materials so as to meet desired performance specifications at the expected conditions of use, the present invention is also directed to post-processing methods and devices for the absorbent polymeric materials. More specifically, the present invention is also directed to processing the polymeric materials (and devices incorporating the polymeric materials) prior to installation or implantation so that, upon installation or implantation, the devices are already equilibrated for the fluid environment in which they will be utilized.

According to this embodiment, following formation of a device including at least one absorptive polymeric material as herein described, the device can be held in a fluid environment similar to that in which it will eventually be installed and utilized. During this holding period, the polymeric material can absorb fluid such that the polymeric material becomes fluid equilibrated and characteristic alterations to the polymeric material upon fluid absorption can take place prior to installation.

For example, according to one embodiment, following formation of the device, the device can be packaged in a fluid-filled container suitable for handling, storing, and shipping the device. During the time the device is held in the container, the polymeric material of the device can become equilibrated with the fluid in the container. At the time the device is removed from the container, which can be immediately prior to installation, the absorbent polymeric material can have absorbed fluid during storage, and the characteristics alterations of the material can have occurred during the time of storage in the fluid such that the material is immediately ready for performance at the desired performance specifications following installation.

In one embodiment, the disclosed devices can be intended for use in vivo. In one particular embodiment, the devices can be load bearing implantable biomedical devices. For instance, the absorbent polymeric material can be a portion of a load-bearing artificial joint such as may be utilized in total joint replacement procedures, including artificial knee joints or artificial hip joints. For example, the acetabular cup of an artificial hip joint or the tibial plateau of an artificial knee joint can be formed of an absorptive polymeric material as herein described. Exemplary biomedical implantable devices, or biomedical devices including implantable portions thereof, encompassed by the disclosed invention can include devices other than those intended for use in a load-bearing applications, however. For example, the devices can be intended for use in a cardiovascular system or in a non-load bearing orthopedic application. A non-limiting exemplary list of such devices can include artificial heart valves, left ventricular assist devices, artificial hearts, vascular stents, reconstructive devices, including structural supports for hard tissue replacement or non-structural void-filling replacement of soft tissue, and the like.

According to this particular embodiment, in which the devices are intended for use in a biologically-based system, the devices can be fluid equilibrated prior to implantation in a suitable biocompatible aqueous solution. For instance, in one embodiment, the devices can be contained in distilled water. In other embodiments, however, the aqueous solution can contain various additives so as to provide a soaking solution closer to that of the intended application environment. For example, in one embodiment, the solution in which an implantable device can be held and equilibrated can be a simulated body fluid.

The soaking solution can contain additives or other materials. For example, the soaking solution can include an antibiotic to prevent bacterial growth within the soaking solution during the period of time the device is contained in the solution. For instance, the soaking solution can include small amounts (e.g., 0.01 to 0.04 g/L) of sodium azide (NaN₃).

When considering implantable devices, the soaking solution can contain biologically active materials that can be useful during storage (as in an antibiotic as mentioned above) and/or following implantation. For instance, the soaking solution can include one or more biologically active additives such as drugs, antimicrobial agents, antiviral agents, nutritional components, growth factors, hormones, antigens, antibodies, and the like that can be absorbed by the polymeric material during the fluid equilibration process. Following implantation, the absorbed materials can be released from the polymeric material through, e.g., diffusion along the concentration gradient in the implantation environment. Thus, the absorptive polymeric materials can function as a delivery device for various biologically active additives.

The invention is not limited to utilization with biomedical devices, however, and in other embodiments devices for use herein can be intended for use in industrial applications. For instance, the disclosed methods and systems can be utilized with industrial polymeric devices such as bearings, flanges, rotors, tubing, gears, and the like.

In one embodiment, the solution in which the device can be fluid equilibrated prior to implant or installation can contain particular materials so as to bring certain characteristics of the absorptive polymeric materials within a targeted range of specifications desired during use of the device. For example, if the electrical characteristics of the polymeric materials have been specified to fall within a certain range during expected use in the fluid environment, the solution in which the device can be held prior to installation can contain predetermined electrolytic materials that can be absorbed during storage and ensure the polymeric materials meet the target specifications at the time of installation. Moreover, the particular additives to the solution, e.g., the electrolytic materials, can be the same or different as those found in the fluid environment in which the device will be installed.

A preferred soaking solution for a device can generally include any predetermined solution that, upon absorption of the soaking solution by the polymeric material, the polymeric material can alter in a predetermined fashion and exhibit the desired characteristics for the fluid environment in which it will be used.

In another embodiment, the device can be intended for use in a non-liquid fluid environment, for example, in a gaseous or vaporous environment. According to this embodiment, as with liquid environments, the device can be contained prior to installation in a fluid environment that can bring the characteristics of the polymeric material of the device within the desired specifications. For example, the device can be intended for use in an environment primarily consisting of a vapor such as steam. According to this embodiment, it can be preferred to package the device in water, and thus bring the polymeric material to a state at which it can function as specified in the steam environment. When considering a gaseous or vaporous working environment, however, the device may optionally be contained in an airtight container including that particular or another suitable gas or vapor.

Similarly, when considering utilization of a device in an environment primarily consisting of an organic fluid, the devices can be contained prior to installation in any fluid that can bring the materials to the desired specifications for use in that fluid environment. In particular, the environment in which the devices are held and equilibrated prior to installation need not be identical to that in which they are eventually intended for use, as long as through the equilibration process that is carried out prior to installation, the absorbent polymeric materials of the devices are brought within the desired specifications for use.

The container in which the device can be fluid equilibrated prior to installation or implantation can be any suitable fluid-tight container as is generally known in the art. In general, the container should be strong enough so as to survive the handling, storing, and shipping demands that could be placed upon it without breaking or developing leaks. For example, the container can be formed of a suitable molded thermoplastic material that can either completely encapsulate the device or optionally can be fitted with a sealable lid. In particular, the container should be of a size to contain the device and enough fluid to provide for fluid absorption by the polymeric material. In those embodiments wherein the container also includes a lid, the lid of the container can be of the same material as the rest of the container or a different material, as desired. For example, the lid can be a formed thermoplastic or foil piece that can be affixed to the container by use of a suitable adhesive.

If desired, the container material can include labels, pictures, instructions, or the like printed or otherwise applied to the surface of the container or the lid. For example, if it is determined that the device should remain in the fluid for a particular amount of time to ensure equilibration, it may be preferred to print a date on the surface of the container to clearly state when the device will be ready for implantation or installation. Similarly, the container may include an expiration date on the surface, for instance in the case of biomedical and/or implantable devices, an expiration date for the ensured sterility of the contents of the container could be included on the surface of the container.

Following processing and storage according to the present invention, a device including the disclosed absorbent polymeric materials can be removed from the container and implanted or installed in a fluid equilibrated state. As such, the devices can immediately function as desired in the fluid environment in which they are used, and can do so while performing at the targeted engineering specifications. Accordingly, the performance characteristics of the device can be consistent from the time of installation, with no ‘break-in’ period necessary during that time when, in the past, the devices became fluid equilibrated. Moreover, the processes and devices of the present invention can enable the utilization of absorbent polymeric materials in applications in which such materials were not considered suitable in the past, and in particular, in demanding applications in which the changes to the materials brought about upon fluid absorption were at least in part responsible for rendering the material characteristics no longer suitable for their desired function.

The invention may be better understood with reference to the Examples, below:

### EXAMPLE 1

Rigid polyurethane formulations were immersed in fresh water and artificial seawater at 40° C. until all samples reached an equilibrium state of fluid absorption. All samples had initial dimensions of 70mm wide×70mm long×35 mm thick (2.75 in.×2.75 in.×1.38 in.). Compression properties of the samples were measured both before immersion and after fluid saturation. Compression tests were done at 22° C., according to ASTM D695-96. Compression speed was 1.3 mm/min (or 0.050 in./min).

Table 1, below, shows the changes of compression properties for the samples upon fluid absorption. The compressive modulus changed considerably after fluid absorption, and the reduction of compressive yield strength was even more pronounced. This indicates that property changes due to fluid absorption may be beneficially considered when designing a polymer product for applications involving load bearing under compression.

TABLE 1

| | | | Distilled | Artificial |
|---|---|---|---|---|
| | | Dry | Water | Seawater |
| Grade | Compressive | 474 (68700) | 280 (40630) | 280 (40600) |
| A | Modulus of | | | |
| | Elasticity, MPa (Psi) | | | |
| | Yield Strength, | 32.3 (4690) | 12.8 (1860) | 13.0 (1890) |
| | Mpa (Psi) | | | |
| Grade | Compressive | 285 (41400) | 150 (21800) | 154 (22300) |
| B | Modulus of | | | |
| | Elasticity, Mpa (Psi) | | | |
| | Yield Strength, | 17.6 (2560) | 5.8 (840) | 7.2 (1050) |
| | Mpa (Psi) | | | |

### EXAMPLE 2

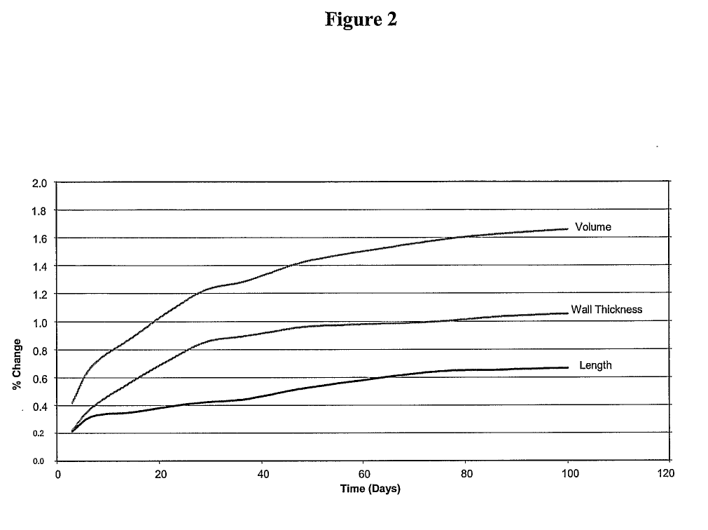

The critical dimensions of cylindrical polyurethane bearing samples, one of the most typical configurations used for bearings, were measured over time after submersion in artificial seawater (3.5 wt % salt water solution). Each bearing tube had an initial inner diameter of 203 mm (8 inches), length of 406 mm (16 inches), and wall thickness of about 12.7 mm (0.5 inches), after being interference fit into a steel housing (about 229 mm or 9″ in inner diameter). When immersed in the seawater, the wall thickness, length, and volume gradually expanded while the inner diameter decreased due to water absorption. In a bearing application, control of the clearance between the shaft and the bearing to provide optimal running clearance for the application is very important. Pre-equilibration of the bearing in the fluid can allow for better control over these critical dimensions. FIG. 2 shows a plot of wall thickness, length, and volume of polymer tube with immersion time in seawater at 40° C. The data were averaged over three bearing samples. After approximately 60 days immersion in the seawater, the bearing dimensions stabilized, indicating that the material had reached its absorption equilibrium balance.

It will be appreciated that the foregoing examples, given for purposes of illustration, are not to be construed as limiting the scope of this invention. Although only a few exemplary embodiments of this invention have been described in detail above, those skilled in the art will readily appreciate that many modifications are possible in the exemplary embodiments without materially departing from the novel teachings and advantages of this invention. Accordingly, all such modifications are intended to be included within the scope of this invention. Further, it is recognized that many embodiments may be conceived that do not achieve all of the advantages of some embodiments, yet the absence of a particular advantage shall not be construed to necessarily mean that such an embodiment is outside the scope of the present invention.

What is claimed is:

1. A method for processing a device intended for use in a fluid environment including a first fluid, the method comprising:
providing a device including an absorptive polymeric material, the polymeric material including a synthetic polymer selected from the group consisting of polyolefins, polyurethanes, silicones, fluoropolymers, polyesters, polyoxymethylenes, polyamides, polyimides, polyamide-imides, polycarbonates, polysulfones, polyphenylene sulfides, polyketones, polybenzimidazole, epoxies, phenolics, bismaleimide, and combinations thereof;
storing the device in a container, a second fluid being held within the container, wherein the polymeric material is capable of absorbing an amount of the second fluid that is less than about 100% of the weight of the polymeric material; and
wherein the polymeric material absorbs at least a portion of the second fluid during the step of storing the device in the container.

2. The method according to claim 1, wherein at least one dimension of the polymeric material changes upon the absorption of the at least a portion of the second fluid by the polymeric material.

3. The method according to claim 1, wherein one or more mechanical characteristics of the polymeric material change upon the absorption of the at least a portion of the second fluid by the polymeric material.

4. The method according to claim 1, wherein one or more electrical characteristics of the polymeric material change upon the absorption of the at least a portion of the second fluid by the polymeric material.

5. The method according to claim 1, wherein the second fluid comprises an additive, wherein the polymeric material absorbs at least a portion of the additive during the step of storing the device in the container.

6. The method according to claim 1, wherein the polymeric material is capable of absorbing an amount of the second fluid that is less than about 20% of the weight of the polymeric material.

7. The method according to claim 1, the method further comprising locating the device in an environment for use, wherein during use, the environment of use is a fluid environment comprising the first fluid.

8. The method according to claim 1, wherein at least one of the first fluid and the second fluid is a gas or a vapor.

9. The method according to claim 1, wherein at least one of the first fluid and the second fluid is a liquid.

10. The method according to claim 1, the method further comprising forming the device.

11. The method according to claim 10, wherein during formation of the device, the polymeric material is formed with a characteristic falling outside of the specifications required for that characteristic for use in the fluid environment.

12. The method according to claim 1, wherein the device is a biocompatible implantable device.

13. A method for storing a device comprising:
providing a device, the device including an absorptive polymeric material that includes a step-growth polymerization polymer;
storing the implantable device in a container, a fluid being held in the container, wherein the polymeric material is capable of absorbing an amount of the fluid that less than about 100% of the weight of the polymeric material; and
wherein the polymeric material absorbs at least a portion of the fluid during the step of storing the device in the container.

14. The method according to claim 13, wherein the device is a biocompatible implantable device.

15. The method according to claim 14, wherein the fluid is a simulated body fluid.

16. The method according to claim 14, the fluid further comprising a biologically active additive, wherein the polymeric material absorbs at least a portion of the additive during the step of storing the device in the container.

17. The method according to claim 13, wherein at least one dimension of the polymeric material changes upon the absorption of the at least a portion of the fluid by the polymeric material.

18. The method according to claim 13, wherein one or more mechanical characteristics of the polymeric material change upon the absorption of the at least a portion of the fluid by the polymeric material.

19. The method according to claim 13, wherein the polymeric material is capable of absorbing an amount of the fluid that is less than about 20% of the weight of the polymeric material.

20. The method according to claim 13, the method further comprising forming the device.

21. The method according to claim 20, wherein during formation of the device, the polymeric material is formed with one or more characteristics falling outside of the specifications required for that characteristic for use in the fluid environment.

22. The method according to claim 13, wherein the polymer is selected from the group consisting of polyurethanes, polyesters, polyoxymethylene, polyamides, polyimides, polyamide-imides, polycarbonates, polysulfones, polyphenylene sulfides, polyketones, polybenzimidazole, epoxies, phenolics, bismaleimide, and combinations thereof.

23. The method according to claim 13, wherein the polymer is a polyurethane.

24. A packaging system for a device intended for use in a fluid environment including a first fluid, the packaging system comprising:
a sealable container defining a volume;
a second fluid held within the volume; and
a device removably locatable within the volume, the device comprising an absorptive polymeric material including a synthetic polymer, the polymeric material being capable of absorbing an amount of the second fluid that is less than about 100% of the -weight of the polymeric material.

25. The packaging system of claim 24, wherein the first fluid and the second fluid are aqueous fluids.

26. The packaging system of claim 24, wherein the first fluid and the second fluid are organic fluids.

27. The packaging system of claim 24, wherein at least one of the first and the second fluids is a gas or a vapor.

28. The packaging system of claim 24, wherein at least one of the first fluid and the second fluid is a liquid.

29. The packaging system of claim 24, wherein the device is intended for use in a load bearing application.

30. The packaging system of claim 24, wherein the device is an implantable device.

31. The packaging system of claim 24, wherein the synthetic polymer is selected from the group consisting of polyolefins, polyurethanes, silicones, fluoropolymers, polyesters, polyoxymethylenes, polyamides, polyimides, polyamide-imides, polycarbonates, polysulfones, polyphenylene sulfides, polyketones, polybenzimidazole, epoxies, phenolics, bismaleimide, and combinations thereof.

32. The packaging system of claim 24, wherein the synthetic polymer is a step-growth polymerization polymer.

33. The packaging system of claim 24, wherein the synthetic polymer is a polyurethane.

34. The packaging system of claim 24, wherein the second fluid comprises an additive.

35. The packaging system of claim 34, wherein the additive is a biologically active additive.

36. The packaging system of claim 34, wherein the biologically active is a drug.

37. The packaging system of claim 34, wherein the additive is an electrolyte.

38. The packaging system of claim 34, wherein the additive is an antibacterial agent.

39. The packaging system of claim 24, wherein the first fluid and the second fluid are the same.
